(19) United States
(12) Patent Application Publication
(10) Pub. No.: US 2008/0208354 A1
(43) Pub. Date: Aug. 28, 2008

(54) Solid Precursor For the Preparation of a Pasty Bone Replacement Material By Admixture of a Liquid

(75) Inventors: Marc Bohner, Grenchen (CH); Giancarlo Rizzoli, Bern (CH)

Correspondence Address: RANKIN, HILL & CLARK LLP, 38210 Glenn Avenue, WILLOUGHBY, OH, 44094-7808, US

(73) Assignee: MATHYS AG BETTLACH, Bettlach (CH)

(21) Appl. No.: 11/996,585

(22) PCT Filed: May 23, 2006
(86) PCT No.: PCT/CH06/00275

§ 371 Date: Jan. 23, 2008

(51) Int. Cl. A61F 2/28; A61K 31/66; A61K 38/29; A61K 38/18; A61P 19/00; A61L 27/00

(52) U.S. Cl. 623/23.62; 514/769; 514/108; 514/12

(57) ABSTRACT

The solid precursor is used for the preparation of a pasty bone replacement material by admixture of a liquid. The precursor comprises calcium-containing ceramic particles and a hydrogel or a substance which can be swelled into a hydrogel; whereby said precursor has been obtained by wet autoclaving and subsequent drying.

### FIELD OF THE INVENTION

The invention relates to a solid precursor for the preparation of a pasty bone replacement material by admixture of a liquid according to the preamble of claim 1.

### DESCRIPTION OF THE PRIOR ART

A number of bone replacement material prepared from solid, dry precursors by admixing of a liquid are known. However, all of the known precursor materials are either non-sterile or are degraded in their molecular structure by the sterilization process. In particular the usual dry autoclaving (e.g. for 120 minutes at 170° C.—WHO 1986) leads to the destruction of most hydrogels used in such bone replacement materials.

Materials which can be injected are also known. For example, hydraulic calcium phosphate cements consist of one or several calcium phosphate powders and an aqueous solution. Upon mixing, a paste is formed. This paste can be injected and hardens within (typically) 5-20 minutes. Unfortunately, the resulting hardened paste is still brittle and can only be resorbed layer-by-layer, i.e. much slower than the pastes described in the present invention. Other injectable pastes consist of non cementitious mixtures of microsized calcium phosphate particles and an aqueous solution. Again, resorption occurs only layer-by-layer. A third alternative is to combine spherical particles (larger than about 0.1 mm) and a low-viscosity hydrogel. These mixtures are injectable, and have a well distributed resorption due to the presence of gaps between the spherical particles, but these mixtures are not kneadable and present a low cohesion.

In the following text, the term “particle” includes any three-dimensional body, regardless of its dimensions, especially the small parts commonly known as “granules” or “grains”. The sphericity S of the particles (or spheric relationship) is defined as the ratio of Dₘₐₓ/Dₘᵢₙ between the largest diameter Dₘₐₓ and the smallest diameter Dₘᵢₙ of the individual particles. Fully spherical particles therefore have a sphericity S=1.00.

This discussion regarding current standards of technology is used only to explain the environment of the invention and does not mean that the standards of technology quoted here were actually published or publicly known at the time of this registration or its priority.

This invention is meant to provide a remedy for this situation. The invention is based on the problem of creating a precursor which overcomes the disadvantages listed above.

The invention solves this task through a precursor which has the characteristics of claim 1.

It is an object of the invention to provide a solid precursor for the preparation of a pasty bone replacement material by admixture of a liquid, whereby said precursor remains stable prior to use and in particular retains its molecular integrity to a high degree.

The advantages of the invention are the following:
Large versatility since the dry precursor can be mixed with many different liquids such as blood, platelet-rich plasma, antibiotic solution;
Very good handling;
Optimum resorption of the kneadable bone replacement material obtained from the precursor; and
Sterility of the bone replacement material obtained without destroying significantly the molecular structure of the hydrogel.

A hydrogel is present when a solid substance is hydrated via a liquid phase, changing and increasing the viscosity of the liquid phase, i.e. jellying or coagulating the liquid phase. Some hydrogels are elastic others are plastic (e.g. sodium hyaluronate). An elastic hydrogel can be destroyed with shear forces, contrary to a plastic (deformable) hydrogel.

The hydrogel matrix can consist of oligomeric or polymeric shares or of a combination of the two.

The calcium-containing ceramic particles and said hydrogel or a substance which can be swelled into a hydrogel may be present as a mixture. Alternatively the hydrogel or a substance which can be swelled into a hydrogel is in powdered form.

Preferably the autoclaving is done in such a manner that it does lead to a loss of molecular weight of the hydrogel of minimum 30% and of maximum 70%. The autoclaving may be performed during 10 to 25 minutes and preferably at a temperature in the range of 110° to 130° C.

The drying of the autoclaved hydrogel may be obtained by the action of dry air, vacuum, freeze-drying and/or a desiccating agent, e.g. P₂O₅.

The hydrogel or the substance which can be swelled into a hydrogel may contain one of the following components: a) polyamino-acids or their derivatives, preferably polylysin or gelatin; b) polysaccharides and their derivatives, preferably glycosaminoglycane or alginate; c) polylipides, fatty acids and their derivatives; d) nucleotides and their derivatives; or a combination of the components as listed in a) through d).

Alternatively the hydrogel or the substance which can be swelled into a hydrogel may contain one of the following components: a) polymethylenoxide or its derivatives; b) polyethylene, polyethylenoxide or their derivatives; c) polypropylene, polypropylenoxide or their derivatives; d) polyacrylate or its derivatives; or a combination of the components as listed in a) through d).

The hydrogel or the substance which can be swelled into a hydrogel may consist of either a glycosaminoglycane or a proteoglycane or a mixture of those two substances. The glycosaminoglycane may be a hyaluronic acid, chondroitinsulfate, dermatansulfate, heparansulfate, heparine or keratansulfate.

In a further embodiment the hydrogel is hyaluronic acid. The hyaluronic acid consists of glucuronic acid and acetylglucosamine which create the disaccharide hyaluronic acid. The hyaluronic acid has a fibrous, non-branched molecular structure and therefore results in highly viscous liquid solutions. The hydrogel may also be in the form of sodium hyaluronate.

In a further embodiment the hydrogel or a substance which can be swelled into a hydrogel is of fully synthetic origin. This eliminates the danger of transferring diseases due to the absence of possible pathogenic agents such as proteins, germs, viruses or bacteria as compared to precursors of natural origin is thus achievable.

Alternatively the hydrogel or the substance which can be swelled into a hydrogel may consist of a biotechnological generated substance.

In a further embodiment the molecular weight of the hydrogel or the substance which can be swelled into a hydrogel is—after sterilization—larger than 300'000 Dalton and preferably larger than 500'000 Dalton. Alternatively the molecular weight of the hydrogel or the substance which can be swelled into a hydrogel may be—after sterilization—smaller than 1050 KDa and preferably in the range of 800-1000 kDa.

In a further embodiment the hydrogel or the substance which can be swelled into a hydrogel is larger than 1'000'000 Dalton and preferably larger than 1'500'000 Dalton.

The precursor may further comprise any drug having an active effect on bone metabolism, preferably osteoinductive substances, drugs against osteoporosis or antimicrobial drugs. Examples for osteoinductive substances are: morphogenetic proteins and growth factors; examples for drugs against osteoporosis are: biphosphonates and parathyroid hormone; an example for an antimicrobial drug is gentamycin sulfate.

In a further embodiment the ceramic particles have at least a partially porous structure. The pore size of the ceramic particles is preferably between 10 nanometers and 500 micrometers. It is also possible to mix ceramic particles with an average diameter between 100 and 250 micrometers and particles with an average diameter between 250 and 500 micrometers or an average diameter between 0.5 and 5.6 mm. This has the advantage that it guarantees the compactness of the bone-replacement material. The interstitial pore volume (pore dead volume) which results from the use of large-grain material can thus be reduced to a minimum. It is also possible to affect the degradation period of the hardened bone-replacement material through the use of ceramic particles of various sizes.

Preferably at least 50% of the ceramic particles have a pore size between 100 and 500 micrometers. This guarantees optimum pore size distribution and the growth of autogenous tissue through the pores.

In a further embodiment the porosity of the ceramic particles is between 60 and 90 percent, preferably between 68 and 84 percent. This ensures that autogenous tissue is able to grow through a larger volume share of ceramic particles.

The average diameter of the ceramic particles is preferably between 100 and 500 micrometers. The advantage of this is the fact that the precursor is compact. In addition, the risk of irritation within the tissue surrounding the particles is practically non-existent, if the diameter of the particles is not smaller than 100 micrometers.

In a further embodiment the ceramic particles consist of a calcium-phosphate which is characterized by a molar Ca/P relationship between 1.0 and 2.0. Preferably the ceramic particles consist of a calcium-phosphate which is characterized by a molar Ca/P relationship between 1.45 and 1.52.

The calcium phosphate may be selected from the following group: Dicalcium-phosphate-dihydrate (CaHPO₄×2H₂O), dicalcium-phosphate (CaHPO₄), alpha-tricalcium-phosphate (alpha-Ca₃(PO₄)₂), beta-tricalcium-phosphate (beta-Ca₃(PO₄)₂), calcium-deficient hydroxyapatite (Ca₉(PO₄)₅(HPO₄)OH), hydroxyapatite (Ca₁₀(PO₄)₆OH)₂), carbonated apatite (Ca₁₀(PO₄)₃(CO₃)₃(OH)₂), flouride-apatite (Ca₁₀(PO₄)₆(F,OH)₂), chloride-apatite (Ca₁₀(PO₄)₆(C₁₀H)₂), whitlockite ((Ca,Mg)₃(PO₄)₂), tetracalcium-phosphate (Ca₄(PO₄)₂O), oxyapatite (Ca₁₀(PO₄)₆O), beta-calcium-pyrophosphate (beta-Ca₂(P₂O₇), alpha-calcium-pyrophosphate, gamma-calcium-pyrophosphate, octo-calcium-phosphate (Ca₈H₂(PO₄)₆×5H₂O).

In a further embodiment the ceramic particles consist of a mixture of different calcium-phosphates. The advantage of such a mixture lies in the control of the resorption period. Due to the differing resorption behaviors of the mixture components, faster bone growth into the cavities of components with faster resorption times can be facilitated.

Alternatively the ceramic particles may consist of a calcium-sulfate, a calcium-carbonate or a mixture of different calcium-phosphates, calcium-sulfates and/or calcium-carbonates. The advantage of such a mixture lies in the control of the resorption period. Due to the differing resorption behaviors of the mixture components, faster bone growth into the cavities of components with faster resorption times can be facilitated.

The specific gravity of the calcium-containing, porous ceramic particles is preferably between 0.5 and 1.0 g/ccm.

In a further embodiment the calcium-containing ceramic particles have a non-spherical shape. “Non-spherical” describes any particle shape which is significantly different from a spherical shape. One variant of the invention uses ceramic particles with an angular shape. “Angular” describes those particles which have individual edges, especially those which are visible with the naked eye, i.e. which are at least 0.1 mm in size. Compared to round particles, these results in an increase to the particle surface, while the average particle diameter remains the same. This causes the adhesive interaction between the particles and the hydrogel to be increased, guaranteeing the mouldability of the bone-replacement material without the need for increasing the quantity of hydrogel used or its concentration.

There is also a special variant whose ceramic particles have a spherical relationship S=Dmax/Dmin between the largest diameter Dmax and the smallest diameter Dmin of the individual particles, which is larger than 1.2 and preferably larger than 1.5. The value of S should be larger than 3 and preferably larger than 5.

At least 60% and typically at least 80% of the ceramic particles should be of a non-spherical shape.

Preferably the maximum amount of humidity in the solid precursor is 3 weight percent.

The preparation of a bone replacement material is obtained by admixing a liquid to the precursor. The following liquids are suitable for that purpose: pure water, sterile demineralized water, an aqueous solution, a sterile saline solution, sterile Ringer solution, serum, blood, bone marrow an antimicrobial drug solution—preferably an antibiotic solution—or a solution containing osteoinductive substances—preferably bone morphogenetic proteins such as BMP2 and BMP7 or growth factors—and/or drugs against osteoporosis—preferably bisphosphonates and parathyroid hormone. The surgeon has the possibility to replace the provided sterile solution with blood or blood extract, bone marrow or bone marrow extract, platelet-rich plasma, a drug solution (e.g. antibiotics, growth factor, drug against osteoporosis) or any other purposeful solution.

The liquid may be sterilized by gamma irradiation or autoclaving.

In a preferred embodiment the ratio between the hydrated hydrogel and the liquid is in the range of 0.001 and 0.200. Higher concentration lead to higher costs and lower concentrations do not lead to the desired “chewing gum” type material. Preferably the ratio between the hydrated hydrogel and the liquid is in the range of 0.03 and 0.09.

In a further embodiment the weight relationship A/B between the hydrated hydrogel and the calcium-containing ceramic particles is larger than 0.2, preferably larger than 0.6. In another embodiment the weight relationship A/B between the hydrated hydrogel and the calcium-containing ceramic particles is smaller than 4, preferably smaller than 2.

The precursor is made available in form of a kit comprising the precursor together with a liquid suitable for admixing to said precursor in order to convert the resulting mixture into a kneadable mass for bone replacement. Preferably the bone replacement material product can be presented to the surgeon as a kit consisting of a sterile powder (b-TCP granules+Na hyal powder) and a sterile liquid, e.g. deionized water or saline solution.

The invention and further developments of the invention are explained in more detail in the following examples:

### EXAMPLE 1

### A) Obtaining a Sterilized Sodium Hyaluronate

An aqueous solution of sodium hyaluronate having a molecular weight of 1428 kDa was autoclaved for 15 minutes at 121° C. By the autoclaving the molecular weight of the sodium hyaluronate was reduced from originally 1400 kDa down to 800 to 1000 kDa (as measure by viscosimetry). The reduction of the molecular weight had no negative effect on the qualities. Drying after wet autoclaving was done in dry air in the presence of P₂O₅ powder under sterile conditions. The sterility was provided by two steam-permeable membranes used to package the material before autoclaving.

### B) Obtaining a Bone-Replacement Material of Putty Consistency

0.12 g of the obtained dried sodium hyaluronate (according to step A), 1.1 g of beta-tricalcium phosphate powder with a size (diameter) in the range of 0.125-0.500 mm and 1.1 g of beta-tricalcium phosphate powder with a size (diameter) in the range of 0.500-0.700 mm were mixed with 2 ml of sterile water with a spatula for 60 second. The beta-tricalcium phosphate powders had a porosity of 60%.

Two minutes after the start of mixing, a slightly elastic and kneadable mass was obtained. This paste was then kneaded to form a long and thin “worm” and inserted into a cancellous bone void resulting from a tibial plateau fracture. The void entry was then closed with the periosteum. Two and a half months after surgery, x-ray pictures demonstrated the presence of new bone in the defect and the start of the resorption process of the b-TCP granules. Full weight bearing could again be applied on the tibia.

### EXAMPLE 2

A mixture of 24 g of porous and angular granules of dicalcium phosphate (DCP) with an approximate size of 500 micrometers and a sphericity degree of S=3.1 and 1.4 g chondroitin sulfate with a molecular weight of MW=535 kDa was sterilized by autoclaving at 121° C. for 15 minutes. Drying after wet autoclaving was done by freeze-drying under sterile conditions.

The sterile dry mixture was mixed with 25 mL of bone marrow aspirated from the pelvic bone of a 10-year old boy. The resulting mixture was kneaded in a sterilized bowl with a sterilized spatula for 1.5 minutes. Two minutes after the start of mixing, a slightly elastic and kneadable mass was obtained. This paste was then inserted into a cyst present in the humerus of the boy. The void entry was then closed with the periosteum. Six weeks after surgery, x-ray pictures demonstrated the presence of new bone in the defect and the start of the resorption process of the DCP granules. No empty void could be detected which could suggest the formation of a new cyst.

### EXAMPLE 3

A mixture of 0.3 g of 0.2-0.3 mm porous and spherical granules of calcium deficient hydroxyapatite and 0.3 g of 0.5-0.7 mm porous and spherical granules of calcium deficient hydroxyapatite was mixed with 50 mg of biotechnologically generated hydroxypropylmethyl cellulose with a molecular weight of 900 kDa.

This mixture was sterilized by autoclaving at 121° C. for 15 minutes. Drying after wet autoclaving was done by the action of dry air under sterile conditions.

Then, 0.1 mL of 5 weight percent gentamicin sulfate solution were added to the dried mixture and thoroughly mixed for 2 minutes. The resulting kneadable material was highly suitable as a plastic bone-replacement material and as a gentamicin delivery system.

### EXAMPLE 4

0.2 g of sodium-alginate (MW=50-500 kDa) and 2.5 g of spherical granules of carbonated apatite with a grain size of 200-300 microns were mixed and sterilized by autoclaving at 121° C. for 15 minutes. Drying after wet autoclaving was done by the action of vacuum under sterile conditions.

Then 2.0 g of sterile Ringer solution were stirred into this dried mixture. This resulted in a kneadable material which was able to be used as a plastic bone-replacement material.

### EXAMPLE 5

0.18 g of sodium hyaluronate (MW=1.1-1.3 million Dalton), 2.5 g of spherical granules of carbonated apatite with a grain size of 200-300 microns and 1.5 g of porous and angular granules of beta-tricalcium-phosphate (β-TCP) with a grain size of 125 to 500 micrometers and a sphericity of S=2.5 were mixed thoroughly and sterilized by autoclaving at 121° C. for 15 minutes. After drying of the sterile mixture 0.5 ml of platelet-rich plasma under sterile conditions an amount of 1.5 ml of sterile deionized water were then stirred into this mixture. After thorough mixing, this resulted in an excellent plastic kneadable material which was able to be used as a plastic bone-replacement material.

### EXAMPLE 6

0.18 g of sodium hyaluronate (MG=1.1-1.3 million Dalton), 1.0 of porous and angular granulates of beta-tricalcium-phosphate (β-TCP) with a grain size of 500 to 700 micrometers and a sphericity degree of S=2.9 and 1.5 g of porous and angular granulates of beta-tricalcium-phosphate (1′-TCP) with a grain size of 125 to 500 micrometers and a sphericity of S=2.5 were mixed thoroughly and sterilized by autoclaving at 121° C. for 15 minutes. After drying of the sterile mixture (under sterile conditions) 2 ml of fresh blood were then stirred into this mixture. After thorough mixing, this resulted in an excellent plastic kneadable material which was able to be used as a plastic bone-replacement material.

### EXAMPLE 7

### A) Manufacture of Powder

A mixture of 6.6 g b-TCP spherical granules (size 0.125-0.500 mm) and 0.27 g Na Hyal powder (MW=1100 kDa) was autoclaved at 121° C. for 15 minutes.

To make sure that autoclaving is effective and that the mixture stays sterile after autoclaving, the mixture was packaged twice in a blister package closed with a paper cover. The latter cover is permeable for steam, but not for germs.

After drying, the double blister package was packaged in an aluminum peel pouch to prevent humidity to decompose Na Hyal during shelf life.

### B) Manufacture of Liquid

6 mL of sterile Ringer solution were filled under aseptic conditions into two blister packages closed with an aluminum-coated membrane. The solution was then gamma irradiated with 25-42 kGray to sterilize it.

### C) Use of the Kit

The product kit consisted of a peel pouch containing the dry component (Na hyal powder—β-TCP granule) and the wet component. The kit was opened by a nurse in the surgical room. The peel pouch containing the dry component was opened above the sterile surgical table to drop the double-blister package onto the latter table. Afterwards, the surgeon opened both blister packages of the dry component, and placed the second (inner) blister package containing the powder/granule mixture on the sterile surgical table. The nurse opened the double blister containing the solution above the sterile surgical table and dropped the inner blister onto the table. The surgeon opened the latter blister, poured the liquid into the blister containing the powder/granules, and using a sterile metallic spatula, mixed the two components for one minute. Afterwards, the surgeon took the resulting paste in the fingers and kneaded it. Two minutes after the start of mixing, the surgeon inserted the paste into a 6 mL cranial defect of a 17 year old boy.

### EXAMPLE 8

### A) Manufacture of powder

6.6 g of spherical b-TCP particles with a diameter of 300+/−50 microns and an apparent density larger than 80% of the theoretical density (3.1 g/cc) and 0.36 g Na Hyal powder (Mw=1429 kDa) were packaged twice in a humidity-permeable blister and autoclaved at 121° C. for 15 minutes. The sample was then freeze-dried until constant weight was reached. The external package was then removed and the inside part (humidity permeable blister) was dropped in a laminar flow bench and packaged in a sterile humidity-impermeable blister.

### B) Manufacture of Liquid

6 mL of sterile distilled water were filled under aseptic conditions into the blister package obtained in step A, and the latter package was closed with an aluminum-coated membrane. The solution was then gamma irradiated with 25-42 kGray to sterilize it.

### C) Use of the Kit

According to example 7

### Methods of Autoclaving Applicable to all of the Examples 1-8

Several methods can be used to sterilize medical products, such as gamma irradiation, heat sterilization (dry air, autoclaving), ethylene oxide sterilization, or plasma sterilization. However, only autoclaving appears to be adequate for powder substances that can be swelled into a hydrogel due to (i) the good homogeneity of the sterilization method, (ii) an absence of toxicity, and (iii) the ability to retain the molecular integrity of the powder substance.

Autoclaving (=steam sterilization) can be performed at various temperatures for various durations. In fact, higher temperatures require shorter sterilization times (logarithmic function). Typically, a temperature of 121° C. and a duration of 15 min are used. At 115° C., a duration of 30 min is used.

1. Solid precursor for the preparation of a pasty bone replacement material by admixture of a liquid, said precursor comprising
a) calcium-containing ceramic particles; and
b) a hydrogel or a substance which can be swelled into a hydrogel; whereby said precursor has been obtained by wet autoclaving and subsequent drying.

2. Precursor according to claim 1, wherein said calcium-containing ceramic particles and said hydrogel or a substance which can be swelled into a hydrogel are present as a mixture.

3. Precursor according to claim 1, wherein the said hydrogel or a substance which can be swelled into a hydrogel is in powdered form.

4. Precursor according to claim 1, wherein the autoclaving does lead to a loss of molecular weight of the hydrogel of minimum 30%.

5. Precursor according to claim 1, wherein the autoclaving does lead to a loss of molecular weight of the hydrogel of maximum 70%.

6. Precursor according to claim 1, wherein the autoclaving is performed during 10 to 25 minutes.

7. Precursor according to claim 1 wherein the autoclaving is performed at a temperature in the range of 110° to 130° C.

8. Precursor according to claim 1, wherein the drying is obtained by the action of dry air, vacuum, freeze-drying and/or a desiccating agent.

9. Precursor according to claim 1, wherein the hydrogel or the substance which can be swelled into a hydrogel contains one of the following components: a) polyamino-acids or their derivatives, preferably polylysin or gelatin; b) polysaccharides and their derivatives, preferably glycosaminoglycane or alginate; c) polylipides, fatty acids and their derivatives; d) nucleotides and their derivatives; or a combination of the components as listed in a) through d).

10. Precursor according to claim 1, wherein the hydrogel or the substance which can be swelled into a hydrogel contains one of the following components: a) polymethylenoxide or its derivatives; b) polyethylene, polyethylenoxide or their derivatives; c) polypropylene, polypropylenoxide or their derivatives; d) polyacrylate or its derivatives; or a combination of the components as listed in a) through d).

11. Precursor according to claim 1, wherein the hydrogel or the substance which can be swelled into a hydrogel consists of either a glycosaminoglycane or a proteoglycane or a mixture of those two substances.

12. Precursor according to claim 11, wherein the glycosaminoglycane is a hyaluronic acid, chondroitinsulfate, dermatansulfate, heparansulfate, heparine or keratansulfate.

13. Precursor according to claim 1, wherein the hydrogel is hyaluronic acid.

14. Precursor according to claim 1, wherein the hydrogel is sodium hyaluronate.

15. Precursor according to claim 1, wherein said hydrogel or a substance which can be swelled into a hydrogel is of fully synthetic origin.

16. Precursor according to claim 1, wherein the hydrogel or the substance which can be swelled into a hydrogel consists of a biotechnological generated substance.

17. Precursor according to claim 1, wherein the molecular weight of the hydrogel or the substance which can be swelled into a hydrogel is—after sterilization—larger than 300,000 Dalton and preferably larger than 500,000 Dalton.

18. Precursor according to claim 1, wherein the molecular weight of the hydrogel or the substance which can be swelled into a hydrogel is—after sterilization—smaller than 1050 KDa.

19. Precursor according to claim 1, wherein the molecular weight of the sterilized hydrogel is in the range of 800-1000 kDa.

20. Precursor according to claim 1, wherein the molecular weight of the hydrogel or the substance which can be swelled into a hydrogel is larger than 1,000,000 Dalton and preferably larger than 500.000 Dalton.

21. Precursor according to claim 1, further comprising any drug having an active effect on bone metabolism, preferably osteoinductive substances, drugs against osteoporosis or antimicrobial drugs.

22. Precursor according to claim 1, wherein the ceramic particles have at least a partially porous structure.

23. Precursor according to claim 22, wherein the pore size of the ceramic particles is between 10 nanometers and 500 micrometers.

24. Precursor according to claim 22, wherein at least 50% of the ceramic particles have a pore size between 100 and 500 micrometers.

25. Precursor according to claim 22, wherein the porosity of the ceramic particles is between 60 and 90 percent, preferably between 68 and 84 percent.

26. Precursor according to claim 1, wherein the average diameter of the ceramic particles is between 100 and 500 micrometers.

27. Precursor according to claim 1, wherein the ceramic particles consist of a calcium-phosphate which is characterized by a molar Ca/P relationship between 1.0 and 2.0.

28. Precursor according to claim 27, wherein the ceramic particles consist of a calcium-phosphate which is characterized by a molar Ca/P relationship between 1.45 and 1.52.

29. Precursor according to one of the claim 27, wherein the calcium phosphate is selected from the following group: Dicalcium-phosphate-dihydrate (CaHPO₄×2H₂O), dicalcium-phosphate (CaHPO₄), alpha-tricalcium-phosphate (alpha-Ca₃(PO₄)₂), beta-tricalcium-phosphate (beta-Ca₃(PO₄)₂), calcium-deficient hydroxyapatite (Ca₉(PO₄)₅(HPO₄)OH), hydroxyapatite (Ca₁₀(PO₄)₆OH)₂), carbonated apatite (Ca₁₀(PO₄)₃(CO₃)₃(OH)₂), flouride-apatite (Ca₁₀(PO₄)₆(F,OH)₂), chloride-apatite (Ca₁₀(PO₄)₆(Cl,OH)₂), whitlockite ((Ca,Mg)₃(PO₄)₂), tetracalcium-phosphate (Ca₄(PO₄)₂O), oxyapatite (Ca₁₀(PO₄)₆O), beta-calcium-pyrophosphate (beta-Ca₂(P₂O₇), alpha-calcium-pyrophosphate, gamma-calcium-pyrophosphate, octo-calcium-phosphate (Ca₈H₂(PO₄)₆×5H₂O).

30. Precursor according to claim 1, wherein the ceramic particles consist of a mixture of different calcium-phosphates.

31. Precursor according to claim 1, wherein the ceramic particles consist of a calcium-sulfate.

32. Precursor according to claim 1, wherein the ceramic particles consist of a calcium-carbonate.

33. Precursor according to claim 1, wherein the ceramic particles consist of a mixture of different calcium-phosphates, calcium-sulfates and/or calcium-carbonates.

34. Precursor according to claim 1, wherein the specific gravity of the calcium-containing, porous ceramic particles is between 0.5 and 1.0 g/ccm.

35. Precursor according to claim 1, wherein the, wherein the calcium-containing ceramic particles have a non-spherical shape.

36. Precursor according to claim 1, wherein the maximum amount of humidity in the solid precursor is 3 weight percent.

37. Bone replacement material obtained by admixing a liquid to the precursor according to claim 1.

38. Bone replacement material according to claim 37, wherein said liquid is pure water, sterile demineralized water, an aqueous solution, a sterile saline solution, sterile Ringer solution, serum, blood, bone marrow an antimicrobial drug solution—preferably an antibiotic solution—or a solution containing osteoinductive substances—preferably bone morphogenetic proteins such as BMP2 and BMP7 or growth factors—and/or drugs against osteoporosis—preferably bisphosphonates and parathyroid hormone.

39. Bone replacement material according to claim 37, wherein said liquid is sterilized by gamma irradiation or autoclaving.

40. Bone replacement material according to claim 1, wherein the ratio between the hydrated hydrogel and the liquid is in the range of 0.001 and 0.200.

41. Bone replacement material according to claim 40, wherein the ratio between the hydrated hydrogel and the liquid is in the range of 0.03 and 0.09.

42. Precursor according to claim 37, wherein the weight relationship A/B between the hydrated hydrogel and the calcium-containing ceramic particles is larger than 0.2.

43. Precursor according to claim 37, wherein the weight relationship A/B between the hydrated hydrogel and the calcium-containing ceramic particles is smaller than 4.

44. Kit comprising the precursor according to claim 1 and a liquid suitable for admixing to said precursor in order to convert the resulting mixture into a kneadable mass for bone replacement.
